(19) United States
(12) Patent Application Publication
(10) Pub. No.: US 2006/0105053 A1
(43) Pub. Date: May 18, 2006

(54) Microsphere filled polymer composites

(75) Inventors: Ryan E. Marx, Rosemount, MN (US); Andrew S. D'Souza, Little Canada, MN (US); Kenneth J. Hanley, Eagan, MN (US); Ronald J. Israelson, Lake Elmo, MN (US); John W. Longabach, Woodbury, MN (US); James M. Nelson, Woodbury, MN (US); Terri A. Shefelbine, St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/280,924

(22) Filed: Nov. 16, 2005

Related U.S. Application Data
(60) Provisional application No. 60/628,335, filed on Nov. 16, 2004.

(51) Int. Cl. A61K 9/50; A61K 9/16

(52) U.S. Cl. 424/490

(57) ABSTRACT

Block copolymers are suitable additives for polymeric composites containing microspheres. The block copolymers have at least one segment that is capable of interacting with the microspheres thereby enhancing the physical characteristics of the composition.

### CROSS-REFERENCE TO RELATED APPLICATION

This application claims the benefit of U.S. Provisional Application No. 60/628,335, entitled “MICROSPHERE FILLED POLYMER COMPOSITES”, filed on Nov. 16, 2004, the entire contents of which are incorporated herein by reference.

### TECHNICAL FIELD

This description relates a polymer composition containing a polymeric matrix, microspheres, and a block copolymer and a method for producing the polymer composition.

### BACKGROUND

In general, microspheres, or other conventional fillers, are often added to polymeric composites to either replace costly polymer components, to enhance specific mechanical characteristics of the overall composites, or both. The enhancements provided by the inclusion of the microspheres are often intended to reduce the warpage and shrinkage or address strength to weight characteristics of the composites. The inclusion of hollow microspheres often provides a reduction in the weight of the composite as well. However, including the microspheres generally results in a trade-off of properties in the final composite. The microspheres may enhance at least one physical property or mechanical characteristic of the composite, while adversely affecting others.

It is conventionally recognized by those of skill in the art that the addition of microspheres to polymeric composites results in decreased mechanical properties such as tensile strength and impact resistance in comparison to the polymer composite without microspheres. The degradation of mechanical properties is generally attributed to the relatively poor adhesion between the polymeric component of the composite and the microspheres.

Silane-based surface treatments on glass and other microspheres have been found to successfully reverse some of the degradation of mechanical properties attributed to poor adhesion between the microsphere surface and the polymeric matrix. Silanes, however, have a low molecular weight, thus providing no entanglement with the polymer. Silanes may be used to recover select mechanical properties, but results vary depending on the type of polymer.

### SUMMARY

The present invention is directed to the use of block copolymers as additives for polymeric composites containing microspheres. The utilization of block copolymers in conjunction with microspheres prevents the generally recognized degradation of mechanical properties of a polymeric composite when microspheres are used alone. The combination of block copolymers with microspheres in a polymeric composite may enhance certain mechanical properties of the composite, such as tensile strength, impact resistance, tensile modulus, and flexural modulus.

The composition of the present invention comprises a polymeric matrix, a plurality of microspheres, and one or more block copolymers. The block copolymers have at least one segment that is capable of interacting with the microspheres. For purposes of the invention, the interaction between the block copolymers and the microspheres is generally recognized as the formation of a bond through either covalent bonding, hydrogen bonding, dipole bonding, or ionic bonding, or combinations thereof. The interaction involving at least one segment of the block copolymer and the microsphere is capable of enhancing or restoring mechanical properties of the polymeric matrix to desirable levels in comparison to polymeric matrices without the block copolymer.

The present invention is also directed to a method of forming a polymeric matrix containing microspheres and one or more block copolymers. The one or more block copolymers are capable of interacting with the microspheres.

The combination of block copolymers with microspheres has applicability in either thermoplastic or thermosetting compositions. The microspheres useful in the inventive composition include all conventional microspheres suitable for use in a polymeric matrix. Preferred microspheres are glass or ceramic, with a most preferred embodiment directed to hollow glass microspheres.

Block copolymers can be tailored for each polymeric matrix, microsphere, or both, adding a broad range of flexibility. In addition, multiple physical properties can be augmented through block design. Block copolymers can be used instead of surface treatments. Alternatively, the block copolymers may be used in tandem with surface treatments.

### DEFINITIONS

For purposes of the present invention, the following terms used in this application are defined as follows:

“Block” refers to a portion of a block copolymer, comprising many monomeric units, that has at least one feature which is not present in the adjacent blocks;

“Compatible mixture” refers to a material capable of forming a dispersion in a continuous matrix of a second material, or capable of forming a co-continuous polymer dispersion of both materials;

“Interaction between the block copolymers and the microspheres” refers to the formation of a bond through either covalent bonding, hydrogen bonding, dipole bonding, or ionic bonding or combinations thereof;

“Block copolymer” means a polymer having at least two compositionally discrete segments, e.g. a di-block copolymer, a tri-block copolymer, a random block copolymer, a graft-block copolymer, a star-branched block copolymer or a hyper-branched block copolymer;

“Random block copolymer” means a copolymer having at least two distinct blocks wherein at least one block comprises a random arrangement of at least two types of monomer units;

“Di-block copolymers or Tri-block copolymers” means a polymer in which all the neighboring monomer units (except at the transition point) are of the same identity, e.g., AB is a di-block copolymer comprised of an A block and a B block that are compositionally different and ABC is a tri-block copolymer comprised of A, B, and C blocks, each compositionally different;

“Graft-block copolymer” means a polymer consisting of a side-chain polymers grafted onto a main chain. The side chain polymer can be any polymer different in composition from the main chain copolymer;

“Star-branched block copolymer” or “Hyper-branched block copolymer” means a polymer consisting of several linear block chains linked together at one end of each chain by a single branch or junction point, also known as a radial block copolymer;

“End functionalized” means a polymer chain terminated with a functional group on at least one chain end; and

“Polymeric matrix” means any resinous phase of a reinforced plastic material in which the additives of a composite are embedded.

### DETAILED DESCRIPTION

The polymeric matrix includes a plurality of microspheres, and one or more block copolymers in a compatible mixture. The block copolymers have at least one segment that is capable of interacting with the microspheres in the compatible mixture. The interaction involving at least one segment of the block copolymer and the microsphere is capable of enhancing or restoring mechanical properties of the polymeric matrix to desirable levels in comparison to polymeric matrices without the block copolymer.

### Polymeric Matrix

The polymeric matrix is generally any thermoplastic or thermosetting polymer or copolymer upon which a block copolymer and microspheres may be employed. The polymeric matrix includes both hydrocarbon and non-hydrocarbon polymers. Examples of useful polymeric matrices include, but are not limited to, polyamides, polyimides, polyethers, polyurethanes, polyolefins, polystyrenes, polyesters, polycarbonates, polyketones, polyureas, polyvinyl resins, polyacrylates, polymethylacrylates, and fluorinated polymers.

One preferred application involves melt-processable polymers where the constituents are dispersed in melt mixing stage prior to formation of an extruded or molded polymer article.

For purposes of the invention, melt processable compositions are those that are capable of being processed while at least a portion of the composition is in a molten state.

Conventionally recognized melt processing methods and equipment may be employed in processing the compositions of the present invention. Non-limiting examples of melt processing practices include extrusion, injection molding, batch mixing, rotation molding, and pultrusion.

Preferred polymeric matrices include polyolefins (e.g., high density polyethylene (HDPE), low density polyethylene (LDPE), linear low density polyethylene (LLDPE), polypropylene (PP)), polyolefin copolymers (e.g., ethylene-butene, ethylene-octene, ethylene vinyl alcohol), polystyrenes, polystyrene copolymers (e.g., high impact polystyrene, acrylonitrile butadiene styrene copolymer), polyacrylates, polymethacrylates, polyesters, polyvinylchloride (PVC), fluoropolymers, liquid crystal polymers, polyamides, polyether imides, polyphenylene sulfides, polysulfones, polyacetals, polycarbonates, polyphenylene oxides, polyurethanes, thermoplastic elastomers, epoxies, alkyds, melamines, phenolics, ureas, vinyl esters or combinations thereof.

The polymeric matrix is included in a melt processable composition in amounts typically greater than about 30% by weight. Those skilled in the art recognize that the amount of polymeric matrix will vary depending upon, for example, the type of polymer, the type of block copolymer, the processing equipment, processing conditions, and the desired end product.

Useful polymeric binders include blends of various polymers and blends thereof containing conventional additives such as antioxidants, light stabilizers, fillers, antiblocking agents, plasticizers, fire retardants, and pigments. The polymeric matrix may be incorporated into the melt processable composition in the form of powders, pellets, granules, or in any other form.

Another preferred polymeric matrix includes pressure sensitive adhesives (PSA). These types of materials are well suited for applications involving microspheres in conjunction with block copolymers. Polymeric matrices suitable for use in PSA's are generally recognized by those of skill in the art and include those fully described in U.S. Pat. Nos. 5,412,031, 5,502,103, 5,693,425, 5,714,548, herein incorporated by reference in their entirety. Additionally, conventional additives with PSA's, such as tackifiers, fillers, plasticizers, pigments fibers, toughening agents, fire retardants, and antioxidants, may also be included in the mixture.

Elastomers are another subset of polymers suitable for use as a polymeric matrix. Useful elastomeric polymeric resins (i.e., elastomers) include thermoplastic and thermoset elastomeric polymeric resins, for example, polybutadiene, polyisobutylene, ethylene propylene copolymers, ethylene-propylene-diene terpolymers, sulfonated ethylene-propylene-diene terpolymers, polychloroprene, poly(2,3-dimethylbutadiene), poly(butadiene-co-pentadiene), chlorosulfonated polyethylenes, polysulfide elastomers, silicone elastomers, poly(butadiene-co-nitrile), hydrogenated nitrile-butadiene copolymers, acrylic elastomers, ethylene-acrylate copolymers.

Useful thermoplastic elastomeric polymer resins include block copolymers, made up of blocks of glassy or crystalline blocks such as, for example, polystyrene, poly(vinyltoluene), poly(t-butylstyrene), and polyester, and the elastomeric blocks such as polybutadiene, polyisoprene, ethylene-propylene copolymers, ethylene-butylene copolymers, polyether ester and the like as, for example, poly(styrene-butadiene-styrene) block copolymers marketed by Shell Chemical Company, Houston, Tex., under the trade designation “KRATON”. Copolymers and/or mixtures of these aforementioned elastomeric polymeric resins can also be used.

Useful polymeric matrices also include fluoropolymers, that is, at least partially fluorinated polymers. Useful fluoropolymers include, for example, those that are preparable (e.g., by free-radical polymerization) from monomers comprising 25 chlorotrifluoroethylene, 2-chloropentafluoropropene, 3-chloropentafluoropropene, vinylidene fluoride, trifluoroethylene, tetrafluoroethylene, 1-hydropentafluoropropene, 2-hydropentafluoropropene, 1,1-dichlorofluoroethylene, dichlorodifluoroethylene, hexafluoropropylene, vinyl fluoride, a perfluorinated vinyl ether (e.g., a perfluoro(alkoxy vinyl ether) such as CF₃OCF₂CF₂CF₂OCF═CF₂, or a perfluoro(alkyl vinyl ether) such as perfluoro(methyl vinyl ether) or perfluoro(propyl vinyl ether)), cure site monomers such as for example, nitrile containing monomers (e.g., CF₂═CFO(CF₂)LCN, CF₂═CFO[CF₂CF(CF₃)O]_{q}(CF₂O)_{y}CF(CF₃)CN, CF₂═CF[OCF₂CF(CF₃)]ᵣO(CF₂)ₜCN, or CF₂═CFO(CF₂)ᵤOCF(CF₃)CN where L=2-12; q=0-4; r=1-2; y=0-6; t=1-4; and u=2-6), bromine containing monomers (e.g., Z-Rf-Ox-CF═CF₂, wherein Z is Br or I, Rf is a substituted or unsubstituted C₁-C₁₂ fluoroalkylene, which may be perfluorinated and may contain one or more ether oxygen atoms, and x is 0 or 1); or a combination thereof, optionally in combination with additional non-fluorinated monomers such as, for example, ethylene or propylene. Specific examples of such fluoropolymers include polyvinylidene fluoride; copolymers of tetrafluoroethylene, hexafluoropropylene and vinylidene fluoride; copolymers of tetrafluoroethylene, hexafluoropropylene, perfluoropropyl vinyl ether, and vinylidene fluoride; tetrafluoroethylene-hexafluoropropylene copolymers; tetrafluoroethyleneperfluoro(alkyl vinyl ether) copolymers (e.g., tetrafluoroethyleneperfluoro(propyl vinyl ether)); and combinations thereof.

Useful commercially available thermoplastic fluoropolymers include, for example, those marketed by Dyneon, LLC, Oakdale, Minn., under the trade designations “THV” (e.g., “THV 220”, “THV 400G”, “THV 500G”, “THV 815”, and “THV 610X”), “PVDF”, “PFA”, “HTE”, “ETFE”, and “FEP”; those marketed by Atofina Chemicals, Philadelphia, Pa., under the trade designation “KYNAR” (e.g., “KYNAR 740”); those marketed by Solvay Solexis, Thorofare, N.J., under the trade designations “HYLAR” (e.g., “HYLAR 700”) and “HALAR ECTFE”.

### Microspheres

Conventional microspheres are employed with the composite of the present invention. The microspheres may be any microsphere generally recognized by those of skill in the art as being suitable for use in a polymer matrix. The utilization of microspheres provides certain mechanical modifications, such as, improvements in strength to density ratios or shrinkage and warpage. The microspheres preferably include glass or ceramic materials and most preferably are hollow glass microspheres. Non-limiting examples of commercially available microsphere include 3M™ Scotchlite™ Glass Bubbles, 3M™ Z-Light™ Spheres Microspheres, and 3M™ Zeeospheres™ Ceramic Microspheres from 3M Company St. Paul, Minn.

### Block Copolymers

The block copolymers are preferably compatible with the polymeric matrix. A compatible mixture refers to a material capable of forming a dispersion in a continuous matrix of a second material, or capable of forming a co-continuous polymer dispersion of both materials. The block copolymers are capable of interacting with the microspheres. In one sense, and without intending to limit the scope of the present invention, applicants believe that the block copolymers may act as a coupling agent to the microspheres in the compatible mixture, as a dispersant in order to consistently distribute the microspheres throughout the compatible mixture, or both.

Preferred examples of block copolymers include di-block copolymers, tri-block copolymers, random block copolymers, graft-block copolymers, star-branched copolymers or hyper-branched copolymers. Additionally, block copolymers may have end functional groups.

Block copolymers are generally formed by sequentially polymerizing different monomers. Useful methods for forming block copolymers include, for example, anionic, cationic, coordination, and free radical polymerization methods.

The block copolymers interact with the microspheres through functional moieties. Functional blocks typically have one or more polar moieties such as, for example, acids (e.g., —CO₂H, —SO₃H, —PO₃H); —OH; —SH; primary, secondary, or tertiary amines; ammonium N-substituted or unsubstituted amides and lactams; N-substituted or unsubstituted thioamides and thiolactams; anhydrides; linear or cyclic ethers and polyethers; isocyanates; cyanates; nitriles; carbamates; ureas; thioureas; heterocyclic amines (e.g., pyridine or imidazole)). Useful monomers that may be used to introduce such groups include, for example, acids (e.g., acrylic acid, methacrylic acid, itaconic acid, maleic acid, fumaric acid, and including methacrylic acid functionality formed via the acid catalyzed deprotection of t-butyl methacrylate monomeric units as described in U.S. Pat. Publ. No. 2004/0024130 (Nelson et al.)); acrylates and methacrylates (e.g., 2-hydroxyethyl acrylate), acrylamide and methacrylamide, N-substituted and N,N-disubstituted acrylamides (e.g., N-t-butylacrylamide, N,N-(dimethylamino)ethylacrylamide, N,N-dimethylacrylamide, N,N-dimethylmethacrylamide), N-ethylacrylamide, N-hydroxyethylacrylamide, N-octylacrylamide, N-t-butylacrylamide, N,N-dimethylacrylamide, N,N-diethylacrylamide, and N-ethyl-N-dihydroxyethylacrylamide), aliphatic amines (e.g., 3-dimethylaminopropyl amine, N,N-dimethylethylenediamine); and heterocyclic monomers (e.g., 2-vinylpyridine, 4-vinylpyridine, 2-(2-aminoethyl)pyridine, 1-(2-aminoethyl)pyrrolidine, 3-aminoquinuclidine, N-vinylpyrrolidone, and N-vinylcaprolactam).

Other suitable blocks typically have one or more hydrophobic moieties such as, for example, aliphatic and aromatic hydrocarbon moieties such as those having at least about 4, 8, 12, or even 18 carbon atoms; fluorinated aliphatic and/or fluorinated aromatic hydrocarbon moieties, such as, for example, those having at least about 4, 8, 12, or even 18 carbon atoms; and silicone moieties.

Non-limiting examples of useful monomers for introducing such blocks include: hydrocarbon olefins such as ethylene, propylene, isoprene, styrene, and butadiene; cyclic siloxanes such as decamethylcyclopentasiloxane and decamethyltetrasiloxane; fluorinated olefins such as tetrafluoroethylene, hexafluoropropylene, trifluoroethylene, difluoroethylene, and chlorofluoroethylene; nonfluorinated alkyl acrylates and methacrylates such as butyl acrylate, isooctyl methacrylate lauryl acrylate, stearyl acrylate; fluorinated acrylates such as perfluoroalkylsulfonamidoalkyl acrylates and methacrylates having the formula H₂C═C(R₂)C(O)O—X—N(R)SO₂R_{f}′ wherein: R_{f}′ is —C₆F₁₃, —C₄F₉, or —C₃F₇; R is hydrogen, C₁ to C₁₀ alkyl, or C₆-C₁₀ aryl; and X is a divalent connecting group. Preferred examples

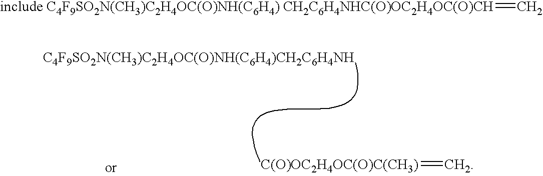

Such monomers may be readily obtained from commercial sources or prepared, for example, according to the procedures in U.S. Pat. Appl. Publ. No. 2004/0023016 (Cernohous et al.), the disclosure of which is incorporated herein by reference.

Other non-limiting examples of useful block copolymers having functional moieties include poly(isoprene-block-4-vinylpyridine); poly(isoprene-block-methacrylic acid); poly(isoprene-block-N,N-(dimethylamino)ethyl acrylate); poly(isoprene-block-2-diethylaminostyrene); poly(isoprene-block-glycidyl methacrylate); poly(isoprene-block-2-hydroxyethyl methacrylate); poly(isoprene-block-N-vinylpyrrolidone); poly(isoprene-block-methacrylic anhydride); poly(isoprene-block-(methacrylic anhydride-co-methacrylic acid)); poly(styrene-block-4-vinylpyridine); poly(styrene-block-2-vinylpyridine); poly(styrene-block-acrylic acid); poly(styrene-block-methacrylamide); poly(styrene-block-N-(3-aminopropyl)methacrylamide); poly(styrene-block-N,N-(dimethylamino)ethyl acrylate); poly(styrene-block-2-diethylaminostyrene); poly(styrene-block-glycidyl methacrylate); poly(styrene-block-2-hydroxyethyl methacrylate); poly(styrene-block-N-vinylpyrrolidone copolymer); poly(styrene-block-isoprene-block-4-vinylpyridine); poly(styrene-block-isoprene-block-glycidyl methacrylate); poly(styrene-block-isoprene-block-methacrylic acid); poly(styrene-block-isoprene-block-(methacrylic anhydride-co-methacrylic acid)); poly(styrene-block-isoprene-block-methacrylic anhydride); poly(butadiene-block-4-vinylpyridine); poly(butadiene-block-methacrylic acid); poly(butadiene-block-N,N-(dimethylamino)ethyl acrylate); poly(butadiene-block-2-diethylaminostyrene); poly(butadiene-block-glycidyl methacrylate); poly(butadiene-block-2-hydroxyethyl methacrylate); poly(butadiene-block-N-vinylpyrrolidone); poly(butadiene-block-methacrylic anhydride); poly(butadiene-block-(methacrylic anhydride-co-methacrylic acid); poly(styrene-block-butadiene-block-4-vinylpyridine); poly(styrene-block-butadiene-block-methacrylic acid); poly(styrene-block-butadiene-block-N,N-(dimethylamino)ethyl acrylate); poly(styrene block-butadiene-block-2-diethylaminostyrene); poly(styrene-block-butadiene-block-glycidyl methacrylate); poly(styrene-block-butadiene-block-2-hydroxyethyl methacrylate); poly(styrene-block-butadiene-block-N-vinylpyrrolidone); poly(styrene-block-butadiene-block-methacrylic anhydride); poly(styrene-block-butadiene-block-(methacrylic anhydride-co-methacrylic acid)); and hydrogenated forms of poly(butadiene-block-4-vinylpyridine), poly(butadiene-block-methacrylic acid), poly(butadiene-block-N,N-(dimethylamino)ethyl acrylate), poly(butadiene-block-2-diethylaminostyrene), poly(butadiene-block-glycidyl methacrylate), poly(butadiene-block-2-hydroxyethyl methacrylate), poly(butadiene-block-N-vinylpyrrolidone), poly(butadiene-block-methacrylic anhydride), poly(butadiene-block-(methacrylic anhydride-co-methacrylic acid)), poly(isoprene-block-4-vinylpyridine), poly(isoprene-block-methacrylic acid), poly(isoprene-block-N,N-(dimethylamino)ethyl acrylate), poly(isoprene-block-2-diethylaminostyrene), poly(isoprene-block-glycidyl methacrylate), poly(isoprene-block-2-hydroxyethyl methacrylate), poly(isoprene-block-N-vinylpyrrolidone), poly(isoprene-block-methacrylic anhydride), poly(isoprene-block-(methacrylic anhydride-co-methacrylic acid)), poly(styrene-block-isoprene-block-glycidyl methacrylate), poly(styrene-block-isoprene-block-methacrylic acid), poly(styrene-block-isoprene-block-methacrylic anhydride-co-methacrylic acid), styrene-block-isoprene-block-methacrylic anhydride, poly(styrene-block-butadiene-block-4-vinylpyridine), poly(styrene-block-butadiene-block-methacrylic acid), poly(styrene-block-butadiene-block-N,N-(dimethylamino)ethyl acrylate), poly(styrene-block-butadiene-block-2-diethylaminostyrene), poly(styrene-block-butadiene-block-glycidyl methacrylate), poly(styrene-block-butadiene-block-2-hydroxyethyl methacrylate), poly(styrene-block-butadiene-block-N-vinylpyrrolidone), poly(styrene-block-butadiene-block-methacrylic anhydride), poly(styrene-block-butadiene-block-(methacrylic anhydride-co-methacrylic acid), poly(MeFBSEMA-block-methacrylic acid) (wherein “MeFBSEMA” refers to 2-(N-methylperfluorobutanesulfonamido)ethyl methacrylate, e.g., as available from 3M Company, Saint Paul, Minn.), poly(MeFBSEMA-block-t-butyl methacrylate), poly(styrene-block-t-butyl methacrylate-block-MeFBSEMA), poly(styrene-block-methacrylic anhydride-block-MeFBSEMA), poly(styrene-block-methacrylic acid-block-MeFBSEMA), poly(styrene-block-(methacrylic anhydride-co-methacrylic acid)-block-MeFBSEMA)), poly(styrene-block-(methacrylic anhydride-co-methacrylic acid-co-MeFBSEMA)), poly(styrene-block-(t-butyl methacrylate-co-MeFBSEMA)), poly(styrene-block-isoprene-block-t-butyl methacrylate-block-MeFBSEMA), poly(styrene-isoprene-block-methacrylic anhydride-block-MeFBSEMA), poly(styrene-isoprene-block-methacrylic acid-block-MeFBSEMA), poly(styrene-block-isoprene-block-(methacrylic anhydride-co-methacrylic acid)-block-MeFBSEMA), poly(styrene-block-isoprene-block-(methacrylic anhydride-co-methacrylic acid-co-MeFBSEMA)), poly(styrene-block-isoprene-block-(t-butyl methacrylate-co-MeFBSEMA)), poly(MeFBSEMA-block-methacrylic anhydride), poly(MeFBSEMA-block-(methacrylic acid-co-methacrylic anhydride)), poly(styrene-block-(t-butyl methacrylate-co-MeFBSEMA)), poly(styrene-block-butadiene-block-t-butyl methacrylate-block-MeFBSEMA), poly(styrene-butadiene-block-methacrylic anhydride-block-MeFBSEMA), poly(styrene-butadiene-block-methacrylic acid-block-MeFBSEMA), poly(styrene-block-butadiene-block-(methacrylic anhydride-co-methacrylic acid)-block-MeFBSEMA), poly(styrene-block-butadiene-block-(methacrylic anhydride-co-methacrylic acid-co-MeFBSEMA)), and poly(styrene-block-butadiene-block-(t-butyl methacrylate-co-MeFBSEMA)).

Generally, the block copolymer should be chosen such that at least one block is capable of interacting with the microspheres. The choice of remaining blocks of the block copolymer will typically be directed by the nature of any polymeric resin with which the block copolymer will be combined.

The block copolymers may be end-functionalized polymeric materials that can be synthesized by using functional initiators or by end-capping living polymer chains, as conventionally recognized in the art. The end-functionalized polymeric materials of the present invention may comprise a polymer terminated with a functional group on at least one chain end. The polymeric species may be homopolymers, copolymers, or block copolymers. For those polymers that have multiple chain ends, the functional groups may be the same or different. Non-limiting examples of functional groups include amine, anhydride, alcohol, carboxylic acid, thiol, maleate, silane, and halide. End-functionalization strategies using living polymerization methods known in the art can be utilized to provide these materials.

Any amount of block copolymer may be used, however, typically the block copolymer is included in an amount in a range of up to 5% by weight.

### Coupling Agents

In a preferred embodiment, the microspheres may be treated with a coupling agent to enhance the interaction between the microspheres and the block copolymer. It is desirable to select a coupling agent that matches or provides suitable reactivity with corresponding functional groups of the block copolymer. Non-limiting examples of coupling agents include zirconates, silanes, or titanates. Typical titanate and zirconate coupling agents are known to those skilled in the art and a detailed overview of the uses and selection criteria for these materials can be found in Monte, S. J., Kenrich Petrochemicals, Inc., “Ken-React® Reference Manual—Titanate, Zirconate and Aluminate Coupling Agents”, Third Revised Edition, March, 1995. The coupling agents are included in an amount of about 1 to 3% by weight.

Suitable silanes are coupled to glass surfaces through condensation reactions to form siloxane linkages with the siliceous filler. This treatment renders the filler more wettable or promotes the adhesion of materials to the glass surface. This provides a mechanism to bring about covalent, ionic or dipole bonding between inorganic fillers and organic matrices. Silane coupling agents are chosen based on the particular functionality desired. For example, an aminosilane glass treatment may be desirable for compounding with a block copolymer containing an anhydride, epoxy or isocyanate group. Alternatively, silane treatments with acidic functionality may require block copolymer selections to possess blocks capable of acid-base interactions, ionic or hydrogen bonding scenarios. Another approach to achieving intimate glass microsphere-block copolymer interactions is to functionalize the glass microsphere with a suitable coupling agent that contains a polymerizable moiety, thus incorporating the material directly into the polymer backbone. Examples of polymerizable moieties are materials that contain olefinic functionality such as styrenic, acrylic and methacrylic moieties. Suitable silane coupling strategies are outlined in Silane Coupling Agents: Connecting Across Boundaries, by Barry Arkles, pg 165-189, Gelest Catalog 3000-A Silanes and Silicones: Gelest Inc. Morrisville, Pa. Those skilled in the art are capable of selecting the appropriate type of coupling agent to match the block copolymer interaction site.

The combination of block copolymers with microspheres in a polymeric composite may enhance certain mechanical properties of the composite, such as tensile strength, impact resistance, tensile modulus, and flexural modulus. In a preferred embodiment, the composition exhibits a maximum tensile strength value within 25% of the maximum tensile strength value of the pure polymer matrix. More preferably, the maximum tensile strength value is within 10% of the maximum tensile strength value of the pure polymer matrix, and even more preferably is within 5%.

The improved physical characteristics render the composites of the present invention suitable for use in many varied applications. Non-limiting examples include, automotive parts (e.g., o-rings, gaskets, hoses, brake pads, instrument panels, side impact panels, bumpers, and fascia), molded household parts, composite sheets, thermoformed parts.

### EXAMPLES

TABLE 1
Materials

| Material | Description |
|---|---|
| PP 3825 | Atofina 3825 - 30 MFI polypropylene, |
| | Available from Atofina Petrochemicals, |
| | Houston, TX |
| PP 1024 | Escorene 1024 12 MFI polypropylene, |
| | commercially available from |
| | ExxonMobil, Irving, TX |
| P(I-MAA) | An AB diblock copolymer, poly[isoprene- |
| | b-methacrylic acid]. Synthesized using a |
| | stirred tubular reactor process as described in |
| | U.S. Pat. No. 6,448,353. Mₙ = 70 kg/mol, |
| | PDI = 1.8, 80/20 PI/MAA by weight |
| P(S-I-MAn) | An ABC triblock copolymer, poly[styrene-b- |
| | isoprene-b-methacrylic anhydride]. Synthesized |
| | using a stirred tubular reactor process as |
| | described in U.S. Pat. No. 6,448,353. Mₙ = 70 kg/mol, |
| | PDI = 1.5, 15/55/30 PS/PI/MAn by weight |
| P(EP-MAn) | An AB diblock copolymer, poly[ethylene-co-propylene- |
| | b-methacrylic acid-co-anhydride]. The precursor |
| | of this block copolymer (poly(isoprene-b-t-butyl |
| | methacrylate) was synthesized using a stirred tubular |
| | reactor process as described in U.S. Pat. No. 6,448,253. |
| | The polymer was hydrogenated to ˜50% and functionalized |
| | according to US20040024130. Mn = 40 kg/mol, |
| | PDI = 1.8, 90/10 PEP/MAn by weight |
| S60HS | 3M ™ Scotchlite ™ Glass Bubbles S60HS |
| | with an average diameter of 30 μm and a 10% isostatic |
| | collapse strength of 19,000 psi, Commercially available |
| | from 3M, St. Paul, MN |
| S80HP | 3M Experimental Glass Bubble S80HP with and |
| | average diameter of 18 μm and a 10% isostatic collapse |
| | strength of 29,000 psi |
| Glass Fiber | Cratec ® 123D chopped glass fiber, Commercially |
| | available from Owens Corning, Toledo, OH |

Batch Composite Formation

A Brabender Torque Rheometer Model PL2100 with a Type 6 mixer head utilizing roller blade mixing paddles was used to compound the microsphere-composites. For all samples, the brabender was heated to 180° C. and mixed at a paddle speed of 50 rpm. The polymeric matrices was initially melted in the brabender and the temperature was allowed to equilibrate. Once a steady melt temperature was reached, microspheres and the block copolymer additive (if used) were added simultaneously. The temperature was allowed to equilibrate once more and the composite was mixed for an additional 5 minutes.

The resultant composite was placed between 2 mil thick untreated polyester liners, which were placed between 2 aluminum plates (⅛ inch thick each) to form a stack. Two shims (1 mm thick) were placed to either side of the mixture between the liners such that upon pressing the assembled stack the mixture would not come into contact with either shim. This stack of materials was placed in a hydraulic press (Wabash MPI model G30H-15-LP). Both the top and bottom press plates were heated to 193° C. The stack was pressed for 1 minute at 1500 psi. The hot stack was then moved to a low-pressure water-cooled press for 30 seconds to cool the stack. The stack was disassembled and the liners were removed from both sides of the film disc that resulted from pressing the mixture.

Physical Property Testing

Tensile bars were stamped out of the composite films produced according to ASTM D1708. The samples were tested on an Instron 5500 R tensile tester (available from Instron Corporation, Canton, Mass.). They were pulled at a rate of 50.8 mm/min in a temperature and humidity controlled room at 21.1° C. and 55% relative humidity. For each sample, 5 specimens were tested and a mean value for the maximum Tensile Strength was calculated.

PP/microsphere composites were made according to the general procedure for Batch Composite Formation. P(EP-MAn) was utilized as a coupling agent and compared to those samples prepared with only microspheres. The compositions and resulting tensile stress measurements are shown in Table 2.

TABLE 2
Example 1 feed compositions and sample tensile strength

| Sample | PP 3825 | | P(EP-MAn) | Max Tensile Stress |
|---|---|---|---|---|
| ID | (g) | S60HS (g) | (g) | (MPa) |
| 1A | Not Processed | 0.0 | 0 | 30.6 |
| 1B | 175.0 | 35.0 | 0 | 20.3 |
| 1C | 175.0 | 35.0 | 5.3 | 26.6 |

As shown in Table 2, the addition of microspheres has a detrimental effect on the tensile strength of PP. Adding just 2.5% of a block copolymer results in an increase in tensile strength of the microsphere-filled composite.

### Example 2

Continuous Composite Formation

Polypropylene composites were compounded using a 19 mm, 15:1 L:D, Haake Rheocord Twin Screw Extruder (commercially available from Haake Inc., Newington, N.H.). The extruder was equipped with a conical counter-rotating screw and the raw materials were dry-blended and fed with an Accurate open helix dry material feeder (commercially available from Accurate Co. Whitewater, Wis.). The extrusion parameters were controlled and experimental data recorded using the Haake RC 9000 control data computerized software (commercially available for Haake Inc., Newington, N.H.). Materials were extruded through a standard 0.05 cm diameter, 4-strand die (commercially available from Haake Inc., Newington, N.H.). The sample compositions are shown in Table 3.

TABLE 3
Example 2 Corn Compositions

| Sample | | Glass | | | | |
|---|---|---|---|---|---|---|
| ID | PP 1024 | fiber | S60HS | S80HP | P(I-MAA) | P(S-I-MAn) |
| 2A | 80.0% | 10.0% | 0.0% | 10.0% | 0.0% | 0.0% |
| Control | | | | | | |
| 2B | 78.0% | 10.0% | 0.0% | 10.0% | 2.0% | 0.0% |
| 2C | 78.0% | 10.0% | 0.0% | 10.0% | 0.0% | 2.0% |
| 2D | 75.0% | 10.0% | 0.0% | 10.0% | 5.0% | 0.0% |
| 2E | 75.0% | 10.0% | 0.0% | 10.0% | 0.0% | 5.0% |
| 2F | 80.0% | 10.0% | 10.0% | 0.0% | 0.0% | 0.0% |
| Control | | | | | | |
| 2G | 78.0% | 10.0% | 10.0% | 0.0% | 2.0% | 0.0% |
| 2H | 78.0% | 10.0% | 10.0% | 0.0% | 0.0% | 2.0% |
| 2I | 75.0% | 10.0% | 10.0% | 0.0% | 5.0% | 0.0% |
| 2J | 75.0% | 10.0% | 10.0% | 0.0% | 0.0% | 5.0% |
| 2K | 80.0% | 10.0% | 5.0% | 5.0% | 0.0% | 0.0% |
| Control | | | | | | |
| 2L | 78.0% | 10.0% | 5.0% | 5.0% | 2.0% | 0.0% |
| 2M | 78.0% | 10.0% | 5.0% | 5.0% | 0.0% | 2.0% |
| 2N | 75.0% | 10.0% | 5.0% | 5.0% | 5.0% | 0.0% |
| 2O | 75.0% | 10.0% | 5.0% | 5.0% | 0.0% | 5.0% |

The resulting pellets were injection molded into tensile bars using a Cincinnati-Milacron-Fanuc Roboshot 110 R injection molding apparatus equipped with a series 16-I control panel (commercially available from Milacron Inc., Batavia, Ohio. The samples were injection molded according to 3M Glass Bubbles Compounding and Injection Molding Guidelines, available at http://www.3m.com/.

Tensile bars for physical property testing were made according to ASTM D1708. The samples were tested on an Instron 5500 R tensile tester (available from Instron Corporation, Canton, Mass.). They were pulled at a rate of 50.8 mm/min in a temperature and humidity controlled room at 21.1° C. and 55% relative humidity. For each sample, 5 specimens were tested and the tensile modulus and tensile stress were calculated. Physical property results for Example 2 are shown in Table 4.

TABLE 4
Physical Property Results for Example 2

| | Tensile Modulus (MPa) | | Max Tensile Stress (MPa) | |
|---|---|---|---|---|
| Sample ID | Mean | S.D. | Mean | S.D. |
| 2A Control | 1587.3 | 111.0 | 34.0 | 0.4 |
| 2B | 1990.6 | 161.4 | 40.2 | 0.8 |
| 2C | 1816.6 | 100.7 | 40.8 | 0.2 |
| 2D | 2087.9 | 209.3 | 44.4 | 1.0 |
| 2E | 1799.3 | 111.1 | 40.3 | 0.7 |
| 2F Control | 1557.0 | 52.6 | 34.1 | 0.5 |
| 2G | 2078.8 | 117.6 | 45.0 | 1.1 |
| 2H | 1811.9 | 83.1 | 41.6 | 0.4 |
| 2I | 2004.3 | 133.0 | 45.4 | 0.7 |
| 2J | 1806.1 | 71.2 | 42.8 | 0.5 |
| 2K Control | 1869.6 | 117.7 | 33.7 | 0.6 |
| 2L | 1959.3 | 122.5 | 36.3 | 0.3 |
| 2M | 1965.4 | 23.3 | 42.8 | 0.7 |
| 2N | 1887.5 | 96.4 | 40.8 | 2.0 |
| 2O | 1782.2 | 144.0 | 41.3 | 0.8 |

With both block copolymer additives, P(I-MAA) and P(S-I-MAn), the max tensile stress and tensile modulus are consistently higher than the controls with no additives. The additive is effective for both sizes of hollow glass microspheres and combinations of the two.

What is claimed is:

1. A composition comprising:
(a) a polymeric matrix;
(b) a plurality of microspheres; and
(c) one or more block copolymers wherein at least one segment of the one or more block copolymers interacts with the microspheres.

2. A composition according to claim 1, wherein the one or more block copolymers are included in an amount of up to 5% by weight.

3. A composition according to claim 1, further comprising one or more of antioxidants, light stabilizers, fillers, antiblocking agents, plasticizers, fire retardants, and pigments.

4. A composition according to claim 1, wherein the surfaces of the microspheres are treated with a coupling agent.

5. A composition according to claim 4, wherein the coupling agent includes zirconates, silanes, or titanates.

6. A composition according to claim 1, wherein the composition exhibits a maximum tensile strength value within 25% of the maximum tensile strength value of the pure polymer matrix.

7. A composition according to claim 1, wherein the block copolymer is selected from one or more of di-block copolymers, a tri-block copolymers, a random block copolymers, a graft-block copolymers, star-branched block copolymers, end-functionalized copolymers, or a hyper-branched block copolymers.

8. A composition according to claim 1, wherein the polymeric matrix is selected from one or more of polyamides, polyimides, polyethers, polyurethanes, polyolefins, polystyrenes, polyesters, polycarbonates, polyketones, polyureas, polyvinyl resins, polyacrylates, fluorinated polymers, and polymethylacrylates.

9. A composition according to claim 1, wherein the at least one segment of the one or more block copolymers is compatible with the polymeric matrix.

10. A composition according to claim 1, wherein microspheres include hollow glass microspheres.

11. A composition comprising:
(a) a plurality of microspheres having surfaces; and
(b) one or more block copolymers wherein at least one segment of the one or more block copolymers is capable of interacting with the microspheres upon application in a polymeric matrix.

12. A method comprising forming a polymeric matrix containing microspheres and one or more block copolymers wherein the one or more block copolymer interacts with the microspheres.
